(19) United States
(12) Patent Application Publication
(10) Pub. No.: US 2013/0189341 A1
(43) Pub. Date: Jul. 25, 2013

(54) GENERATION OF PHOTORECEPTORS FROM HUMAN RETINAL PROGENITOR CELLS USING POLYCAPROLACTONE SUBSTRATES

(71) Applicants: Caio Regatieri, Boston, MA (US); Petr Y. Baranov, Somerville, MA (US); Michael J. Young, Gloucester, MA (US)

(72) Inventors: Caio Regatieri, Boston, MA (US); Petr Y. Baranov, Somerville, MA (US); Michael J. Young, Gloucester, MA (US)

(21) Appl. No.: 13/356,073

(22) Filed: Jan. 23, 2012

(51) Int. Cl. A61K 9/70; A61F 2/16; C12Q 1/02; A61K 35/44; A61P 27/02

(52) U.S. Cl. 424/426; 424/443; 424/93.7; 435/29; 623/6.63

(57) ABSTRACT

The present invention relates to biocompatible compositions for transplantation into a sub-retinal space of the human eye. The compositions include a biodegradable polyester film, preferably a polycaprolactone (PCL) film, and a layer of human retinal progenitor cells. The compositions of the invention can be used as scaffolds for the treatment a number of ocular diseases, including retinitis pigmentosa and age-related macular degeneration.

### BACKGROUND OF THE INVENTION

The degeneration of the human retina, either as a result of trauma, age or disease, can result in permanent visual loss and affect millions of people worldwide. Degenerative conditions include, for instance, retinitis pigmentosa, age-related macular degeneration and diabetic retinopathy. These conditions are characterized by the progressive death of light sensing photoreceptor cells of the retina, and are the leading causes of incurable blindness in the western world. As the intrinsic regenerative capacity of the human retina is extremely limited, the only viable treatment option for people suffering from photoreceptor cell loss is cellular replacement.

One strategy for replacing photoreceptor cells is to transplant retinal tissue from healthy donors to the retina of the diseased host. While the results of such strategies have been encouraging in terms of tissue graft survival, the problems of the graft and host tissue remain daunting. Laboratory studies have consequently focused on multipotent stem cells (also variously referred to as progenitor cells, immature cells, precursor cells, undifferentiated cells or proliferative cells) for transplantation and differentiation.

The isolation of true stem cells from the neuroretina, particularly cells able to differentiate into functional photoreceptor cells both in vitro and in vivo, has proven elusive. Putative retinal stem cells derived from the ciliary marginal zone pigment epithelial layer are described in U.S. Pat. No. 6,117,675. While these cells are said to be capable of proliferating in the absence of growth factors, there is very limited evidence that these cells are capable of integrating into a host retina and differentiating into functional mature cells in vivo. Moreover, these cells fail to differentiate into viable photoreceptors. Indeed, the existence of these “stem cells” remains controversial.

Retinal progenitor cells, isolated from the fetal retina, can be expanded in vitro, and after transplantation to retinal degenerative hosts, are capable of migrating into, integrating with, and forming new functioning photoreceptors. See, for instance, commonly assigned U.S. Pat. No. 7,514,259, directed to neuroretina-derived photoreceptor cells which are capable of repopulating a human retina. These cells are derived from neural retinal tissue by removing the ciliary marginal zone and the optic nerve to eliminate contamination, and can be obtained from pre- and post-natal tissue.

A significant obstacle for deploying this technology in the clinic is the inability of human retinal progenitor cells to generate large numbers of photoreceptors during differentiation, both in vitro and in vivo following cell transplantation. According to studies conducted in vitro and ex vivo in animal models, only a small percentage of transplanted cells integrate into the host retina and remain viable. The remaining cells either experience cell death, remain undifferentiated, or migrate from the transplantation site. This represents a significant obstacle for photoreceptors intended for use in the clinic, as well as in drug screening and testing applications since there are currently no other available high output and reproducible methods for generating mammalian photoreceptors.

Other approaches for promoting the differentiation of human retinal progenitor cells into photoreceptors utilize growth factors, such as IGF, Dkk-1 and Noggin, media supplements, such as N2 and B27, serum (fetal bovine serum) or serum replacement, and undefined extracellular matrices, such as Matrigel™ and Stellgent™. These approaches are capable of generating only a limited number of photoreceptors from human retinal progenitor cells, and such limited numbers are insufficient to achieve the desired outcome in clinical applications.

Transplantation of viable retinal stem cells into a human retina can also be problematic. It has been shown that injecting suspensions of retinal progenitor cells directly into the retina can result in massive transplant cell losses due to efflux and cell death. For instance, some recent studies have shown that less than 0.5% of cells injected by bolus injection techniques are actually capable of migrating into the retina, while other studies have shown that attempts to deliver-brain-derived neurons into the subretinal space resulted in approximately 90% cell death during the injection process alone.

Retinal tissue engineering strategies involving the use of scaffolds for cell transplantation have also been attempted. These scaffolds are micromachined from biocompatible polymers, such as polymethyl methacrylate (PMMA) and polyglycerol sebacate (PGS), to form thin substrates for depositing cells. The advantage of biocompatible polymers is that they provide temporary scaffolding that can be absorbed by the host and result in de novo tissue. Relatively thin scaffolds of less than 50 μm can be generated by micromaching techniques involving a two-step process of photolithography and reactive ion etching. While these techniques represent an improvement over bolus injections, the use of PGS and PMMA as scaffolding materials has not proven to be particularly successful for facilitating the differentiation of the retinal progenitor cells into photoreceptors following transplantation, which is critical for clinical acceptance. See Tao et al., Lab on a Chip, Royal Society of Chemistry, pp 1-10 (2007); and Redenti et al., Biomaterials, 30. pp 3405-3414 (2009), the respective disclosures of which are incorporated herein by reference.

In view of the aforementioned, as well as the importance of human retinal progenitor cells for clinical evaluation and use, it will readily be appreciated that a need exists to improve the ability to deliver cells into the subretinal space, and to improve the ability of such cells to differentiate and reproduce in vitro while maintaining plasticity properties in vivo. These and other objectives of the invention will be clear from the following description.

### SUMMARY OF THE INVENTION

The invention is directed to compositions comprising a biodegradable, biocompatible polyester film substrate having retinal progenitor cells deposited on the surface of the film. The cells are deposited onto the substrate and adhere to at least a portion of the film surface, thereby providing for enhanced cell differentiation, and the generation of photoreceptor cells (both rods and cones). The progenitor cells can be cultured and differentiate into retinal-specific photoreceptors which can be used for treating retinal disorders by implantation into a subretinal space of the eye with or without the polyester film. The combination of the progenitor cells and films can be used as a tissue scaffold for implantation in a patient. Alternatively, one can also use the polymer scaffold as a means to pre-differentiate progenitor cells into more mature cells for use in retinal transplantation. The compositions and cells of the invention can also be used in drug discovery and in vitro testing applications to identify promising therapeutic targets using cell based assays.

The biodegradable and biocompatible polyester which can be used in the practice of this invention is capable of supporting retinal progenitor cells for growth and differentiation. Preferably, the biodegradable polyester is selected from the group consisting of polylactic acid (PLA), polycaprolactone (PCL), polyesteramide (PEA), polyhydroxybutyrate (PHB), and derivatives and mixtures thereof. Polycaprolactone (PCL) is an especially preferred polyester. Thin films prepared from the polyester of the invention can typically have a thickness of from about 1 micron (μm) to about 50 microns, preferably from about 1 micron to about 10 microns, and most preferably about 5 microns.

The retinal progenitor cells of the invention can be obtained from human post-natal human adult retinal tissue sources, and from fetal retina. According to the invention, human retinal progenitor cells are obtained from viable neuroretinal source tissue, such as the retinal neurosphere. Although these retinal progenitor cells have the potential to differentiate into six neuronal cell types, the photoreceptor cell is the mature cell type desired in the present invention.

The retinal progenitor cells can be deposited or plated directly onto the polymer film, preferably as a mono-layer of cells. The film surface can be smooth or textured to provide improved adherence of the cells. The texturing can, for instance, include the formation of submicron groves or submicron posts as part of the polymer surface topography during the film fabrication process. In one embodiment, an intermediate coating can be provided on the polymer film prior to the deposition of the retinal progenitor cells. Such coatings can include poly-D-lysine, poly-L-lysine, fibronectin, laminin, collagen I, collagen IV, vitronectin and matrigel.

The compositions according to the invention useful for the treatment of retinal diseases upon transplantation into a diseased eye. Thus, the invention provides a method to obtain a population of mutipotent retinal progenitor cells on a support substrate polyester film in vitro suitable for in vivo transplantation into a host recipient. In one aspect, the population of multipotent progenitor cells is substantially homogeneous, e.g. clonally expanded.

The compositions and cells of the invention have therapeutic uses and can be autologous or allogeneic to the host patient or recipient. The compositions and cells of the invention can also be used for drug discovery and testing. Because the retinal progenitor cells are capable of differentiating into photoreceptor cells, they are useful to replace or repair photoreceptor tissue in a patient and, e.g., for the treatment of degenerative diseases of the eye such as retinitis pigmentosa, age-related macular degeneration and diabetic retinopathy.

The foregoing embodiments and aspects of the invention are illustrative only, and are not meant to restrict the spirit and scope of the claimed invention.

### BRIEF DESCRIPTION OF THE DRAWINGS

The foregoing and other advantages and features of the invention will become apparent upon reading the following detailed description with reference to the accompanying figures and drawings.

FIGS. 1A, 1B and 1C are representations of the surface features of the polyester films of the invention showing, respectively, a smooth film, a film with a micro-grooved surface, and a film with micro-posts on its surface.

FIGS. 2A, 2B and 2C are scanning electronic microscopy images of PCL films having plated retinal progenitor cells on the film surface, with smooth, micro-grooves and micro-post film surfaces, respectively.

FIG. 3 is a graph depicting retinal progenitor cell proliferation over a period of seven days for cells grown on films having smooth, micro-grooves and micro-post surfaces.

FIG. 4 is a bar graph showing proliferative marker Ki67 for cells under control conditions (P5), and for cells plated on PCL films having smooth, micro-grooved and micro-post surfaces.

FIGS. 5A-5D are a series of bar graphs showing, respectively, the differentiation cell markers CRX, Recoverin, Rhodopsin and Opsin Blue, for control cells (P5) and cells plated on PCL films having smooth, micro-grooved and micro-post surfaces.

FIGS. 6A-6C are a series of bar graphs showing, respectively, the sternness cell markers PAX6, cMyc and SOX2, for control cells (P5) and cells plated on PCL films having smooth, micro-grooved and micro-post surfaces.

### DETAILED DESCRIPTION OF THE EMBODIMENTS

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods, devices, and materials are now described. All technical and patent publications cited herein are incorporated herein by reference in their entirety. Nothing herein is to be construed as an admission that the invention is not entitled to antedate such disclosure by virtue of prior invention.

The practice of the present invention will employ, unless otherwise indicated, conventional techniques of tissue culture, immunology, molecular biology, microbiology, cell biology and recombinant DNA, which are within the skill of the art. See, e.g., Sambrook and Russell eds. (2001) Molecular Cloning: A Laboratory Manual, 3^{rd} edition; the series Ausubel et al. eds. (2007) Current Protocols in Molecular Biology; the series Methods in Enzymology (Academic Press, Inc., N.Y.); MacPherson et al. (1991) PCR 1: A Practical Approach (IRL Press at Oxford University Press); MacPherson et al. (1995) PCR 2: A Practical Approach; Harlow and Lane eds. (1999) Antibodies, A Laboratory Manual; Freshney (2005) Culture of Animal Cells: A Manual of Basic Technique, 5^{th} edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; Immobilized Cells and Enzymes (IRL Press (1986)); Perbal (1984) A Practical Guide to Molecular Cloning; Miller and Calos eds. (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); and Herzenberg et al. eds (1996) Weir's Handbook of Experimental Immunology.

All numerical designations, e.g., pH, temperature, time, concentration, and molecular weight, including ranges, are approximations which are varied (+) or (−) by increments of 1.0 or 0.1, as appropriate. It is to be understood, although not always explicitly stated, that all numerical designations are preceded by the term “about”. It also is to be understood, although not always explicitly stated, that the reagents described herein are merely exemplary and that equivalents of such are known in the art.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into subranges as discussed above.

As used in the specification and claims, the singular form “a”, “an” and “the” include plural references unless the context clearly dictates otherwise. For example, the term “a pharmaceutically acceptable carrier” includes a plurality of pharmaceutically acceptable carriers, including mixtures thereof.

As used herein, the term “comprising” is intended to mean that the compositions and methods include the recited elements, but do not exclude others. “Consisting essentially of” when used to define compositions and methods, shall mean excluding other elements of any essential significance to the combination for the intended use. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions of this invention. Embodiments defined by each of these transitional terms are within the scope of this invention.

A “host” or “patient” of this invention is an animal such as a mammal, or a human. Non-human animals subject to diagnosis or treatment are those in need of treatment such as for example, simians, murines, such as, rats, mice, canines, such as dogs, leporids, such as rabbits, livestock, sport animals, and pets.

The term “isolated” means separated from constituents, cellular and otherwise, in which the cell, tissue, polynucleotide, peptide, polypeptide, protein, antibody or fragment(s) thereof, which are normally associated in nature. For example, an isolated polynucleotide is separated from the 3′ and 5′ contiguous nucleotides with which it is normally associated in its native or natural environment, e.g., on the chromosome. As is apparent to those of skill in the art, a non-naturally occurring polynucleotide, peptide, polypeptide, protein, antibody or fragment(s) thereof, does not require “isolation” to distinguish it from its naturally occurring counterpart. An isolated cell is a cell that is separated form tissue or cells of dissimilar phenotype or genotype.

As used herein, “stem cell” defines a cell with the ability to divide for indefinite periods in culture and give rise to specialized cells. At this time and for convenience, stem cells are categorized as somatic (adult) or embryonic. A somatic stem cell is an undifferentiated cell found in a differentiated tissue that can renew itself (clonal) and (with certain limitations) differentiate to yield all the specialized cell types of the tissue from which it originated. An embryonic stem cell is a primitive (undifferentiated) cell from the embryo that has the potential to become a wide variety of specialized cell types. An embryonic stem cell is one that has been cultured under in vitro conditions that allow proliferation without differentiation for months to years. Pluripotent embryonic stem cells can be distinguished from other types of cells by the use of marker including, but not limited to, Oct-4, alkaline phosphatase, CD30, TDGF-1, GCTM-2, Genesis, Germ cell nuclear factor, SSEA1, SSEA3, and SSEA4. The term “stem cell” also includes “dedifferentiated” stem cells, an example of which is a somatic cell which is directly converted to a stem cell, i.e. reprogrammed. A clone is a line of cells that is genetically identical to the originating cell; in this case, a stem cell.

The term “propagate” or “proliferate” means to grow or alter the phenotype of a cell or population of cells. The term “growing” or “expanding” refers to the proliferation of cells in the presence of supporting media, nutrients, growth factors, support cells, or any chemical or biological compound necessary for obtaining the desired number of cells or cell type. In one embodiment, the growing of cells results in the regeneration of tissue. In yet another embodiment, the tissue is comprised of cardiomyocytes.

The term “culturing” refers to the in vitro propagation of cells or organisms on or in media of various kinds. It is understood that the descendants of a cell grown in culture may not be completely identical (i.e., morphologically, genetically, or phenotypically) to the parent cell. By “expanded” is meant any proliferation or division of cells. “Clonal proliferation” refers to the growth of a population of cells by the continuous division of single cells into two identical daughter cells and/or population of identical cells.

As used herein, the “lineage” of a cell defines the heredity of the cell, i.e. its predecessors and progeny. The lineage of a cell places the cell within a hereditary scheme of development and differentiation.

“Differentiation” describes the process whereby an unspecialized cell acquires the features of a specialized cell such as a heart, liver, or muscle cell. “Directed differentiation” refers to the manipulation of stem cell culture conditions to induce differentiation into a particular cell type or phenotype. “Dedifferentiated” defines a cell that reverts to a less committed position within the lineage of a cell. As used herein, the term “differentiates or differentiated” defines a cell that takes on a more committed (“differentiated”) position within the lineage of a cell.

“Retinal progenitor cells”, or “neuroretina-derived retinal stem cells”, or “retinal stem cells”, as those terms are used herein, are synonymous and mean isolated viable stem cells derived from neuroretinal tissue. The point of origin of these cells is one factor that distinguishes them from non-neural retinal cells, such as pigmented cells of the retinal pigment epithelium, the ciliary body or the iris. The cells of the invention are further distinguished by an inability to proliferate in the absence of growth factors. The cells of the invention can derived from either pre-natal or post-natal sources, and are multipotent, meaning they are capable of self-renewal and retina-specific differentiation into photoreceptors. Such cells are more particularly described in U.S. Pat. No. 7,514,259, the disclosure of which is incorporated by reference herein in its entirety. The retinal stem cells or retinal progenitor cells of the invention are capable of: (a) self-renewal in vitro; (b) differentiating into neurons and astrocytes (but not oligodendrocytes); (c) integrating into the neuroretina following transplantation to the posterior segment of the eye; and (d) differentiation into photoreceptor cells when grafted onto a retinal explant, or into the mature eye of a recipient. Importantly, there is evidence that differentiation is enhanced, rather than inhibited, by transplantation into the diseased retina (as compared to the normal, healthy retina).

As used herein in connection with the retinal progenitor cells of the invention, the term “multipotency”, means the ability of the retinal progenitor cells to proliferate and four mature retinal cell types, particularly photoreceptor cells.

“Substantially homogeneous” describes a population of cells in which more than about 50%, or alternatively more than about 60%, or alternatively more than 70%, or alternatively more than 75%, or alternatively more than 80%, or alternatively more than 85%, or alternatively more than 90%, or alternatively, more than 95%, of the cells are of the same or similar phenotype. Phenotype can be determined by a pre-selected cell surface marker or other marker, e.g. myosin or actin or the expression of a gene or protein,

As used herein, the terms “treating,” “treatment” and the like are used herein to mean obtaining a desired pharmacologic and/or physiologic effect. The effect can be prophylactic in terms of completely or partially preventing a disorder or sign or symptom thereof, and/or can be therapeutic in terms of a partial or complete cure for a disorder and/or adverse effect attributable to the disorder. Examples of “treatment” include but are not limited to: preventing a disorder from occurring in a subject that may be predisposed to a disorder, but has not yet been diagnosed as having it; inhibiting a disorder, i.e., arresting its development; and/or relieving or ameliorating the symptoms of disorder, e.g., macular degeneration. As is understood by those skilled in the art, “treatment” can include systemic amelioration of the symptoms associated with the pathology and/or a delay in onset of symptoms such as chest pain. Clinical and sub-clinical evidence of “treatment” will vary with the pathology, the individual and the treatment.

The term “biocompatible” means the ability of a biomaterial to perform its desired function with respect to a medical therapy, without eliciting undesirable local or system effects in the recipient or beneficiary of the therapy, but generating an appropriate cellular or tissue response in a specific situation, and optimizing the clinically relevant performance or therapy. “Biocompatibility of an implanted medical device is the capability of the device to exist in the body in harmony with tissue without causing deleterious changes.

A “biocompatible scaffold” refers to a scaffold or matrix for tissue-engineering purposes with the ability to perform as a substrate that will support the appropriate cellular activity to generate the desired tissue, including the facilitation of molecular and mechanical signaling systems, without eliciting any undesirable effect in those cells or inducing any undesirable local or systemic responses in the eventual host. In other embodiments, a biocompatible scaffold is a precursor to an implantable device which has the ability to perform its intended function, with the desired degree of incorporation in the host, without eliciting an undesirable local or systemic effects in the host. Biocompatible scaffolds are described in U.S. Pat. No. 6,638,369.

A “biodegradable polymer” is a non-toxic polymer capable of maintaining its mechanical integrity until it degrades, and which is capable of a controlled rate of degradation. A biodegradable polymer is a polymer which does not illicit an immune response in an organism, such as when used as an implant substrate, and the products of polymer degradation in the organism are non-toxic.

A “composition” is intended to mean a combination of active agent, cell or population of cells and another compound or composition, inert (for example, a detectable agent or label) or active, such as a biocompatible scaffold.

A “pharmaceutical composition” is intended to include the combination of an active agent with a carrier, inert or active such as a biocompatible scaffold, making the composition suitable for diagnostic or therapeutic use in vitro, in vivo or ex vivo.

As used herein, the term “pharmaceutically acceptable carrier” encompasses any of the standard pharmaceutical carriers, such as a phosphate buffered saline solution, water, and emulsions, such as an oil/water or water/oil emulsion, and various types of wetting agents. The compositions also can include stabilizers and preservatives. For examples of carriers, stabilizers and adjuvants, see Martin, Remington's Pharm. Sci., 15th Ed. (Mack Publ. Co., Easton (1975)).

An “effective amount” is an amount sufficient to effect beneficial or desired results. An effective amount can be administered in one or more administrations, applications or dosages.

### Biodegradable Polymer films, Retinal Progenitor Cells, Biocompatible Scaffolds

The invention relates to a biocompatible composition comprising a biodegradable polyester film support for retinal progenitor cells. The biodegradable polyester can be any biodegradable polyester suitable for use as a substrate or scaffold for supporting the proliferation and differentiation of retinal progenitor cells. The polyester should be capable of forming a thin film, preferably a micro-textured film, and should be biodegradable if used for tissue or cell transplantation.

Suitable biodegradable polyesters for use in the invention include polylactic acid (PLA), polylactides, polyhydroxyalkanoates, both homopolymers and co-polymers, such as polyhydoxybutyrate (PHB), polyhydroxybutyrate co-hydroxyvalerate (PHBV), polyhydroxybutyrate co-hydroxyhexanote (PHBHx), polyhydroxybutyrate co-hydroxyoctonoate (PHBO) and polyhydroxybutyrate co-hydroxyoctadecanoate (PHBOd), polycaprolactone (PCL), polyesteramide (PEA), aliphatic copolyesters, such as polybutylene succinate (PBS) and polybutylene succinate/adipate (PBSA), aromatic copolyesters. Both high and low molecular weight polyesters, substituted and unsubstituted polyester, block, branched or random, and polyester mixtures and blends can be used. Preferably the biodegradable polyester is polycaprolactone (PCL).

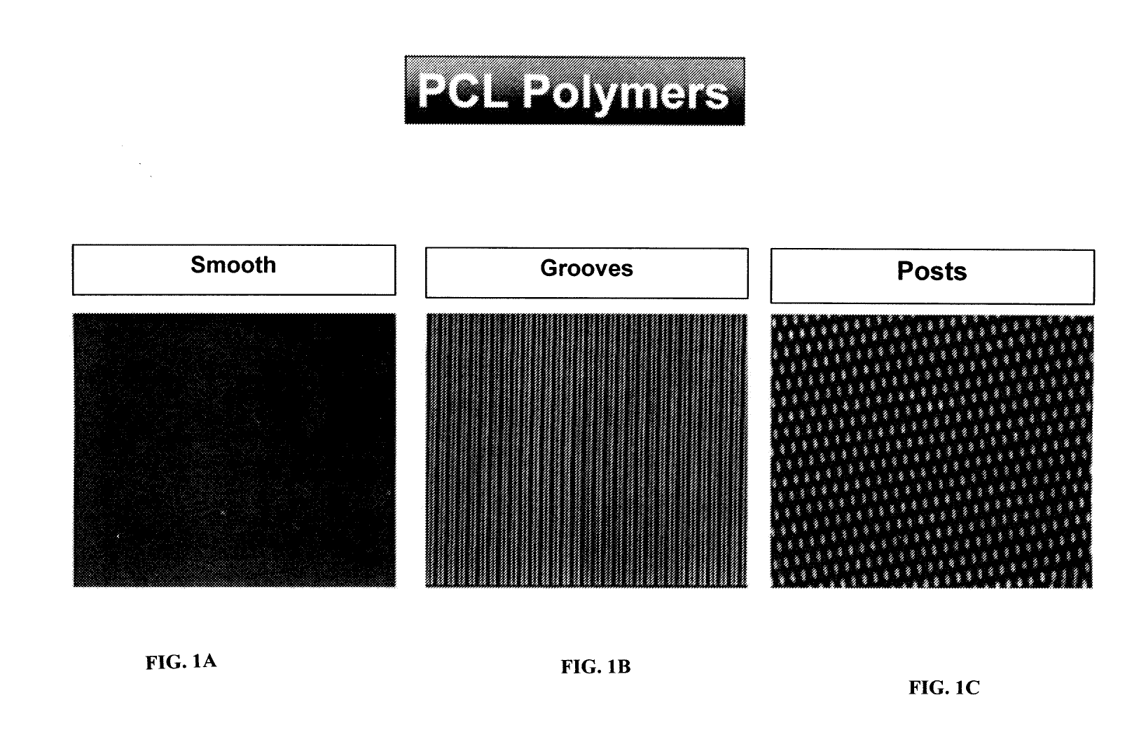

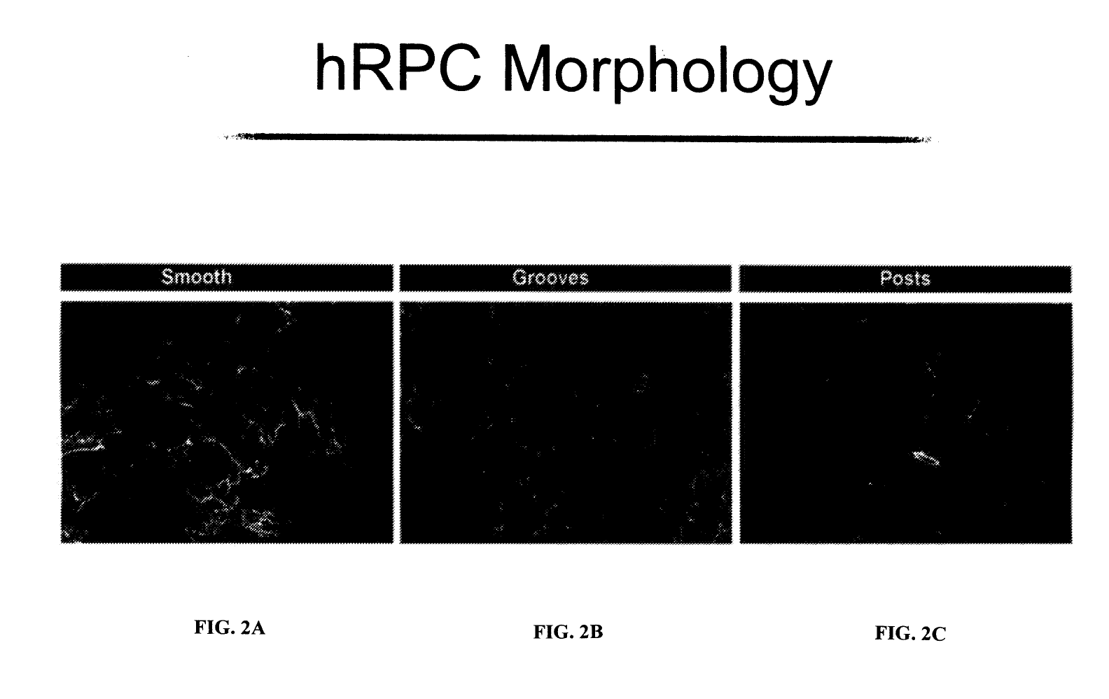

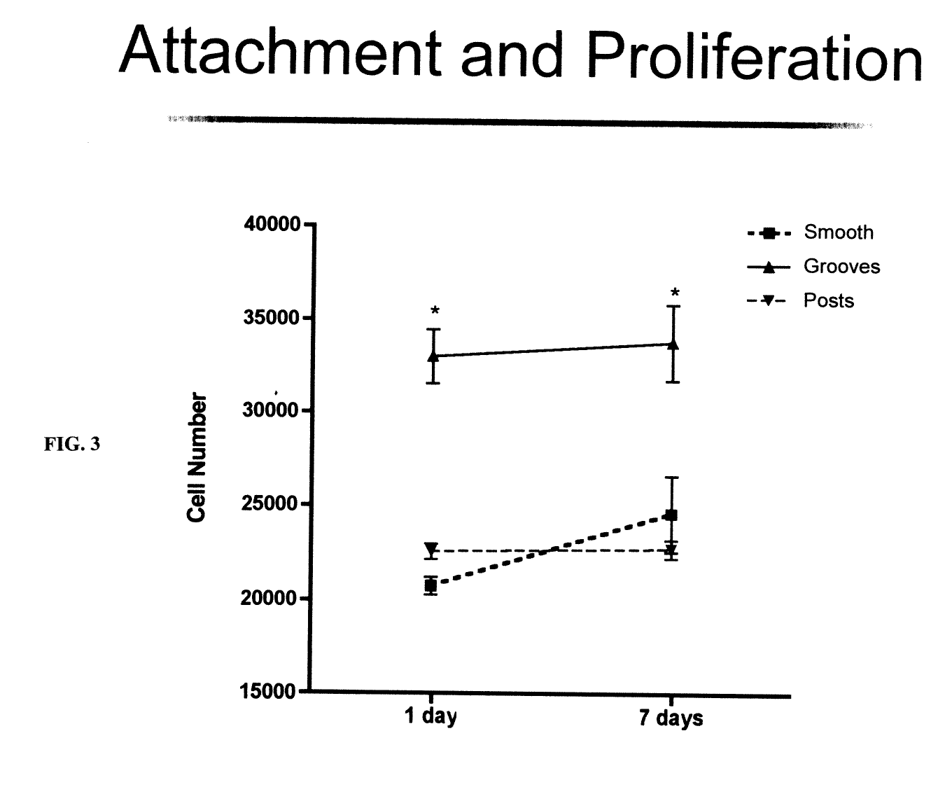

The biodegradable polyester can be formed into a thin film using known techniques. The film thickness is advantageously from about 1 micron (μm) to about 50 microns (μm), and preferably about 5 μm in thickness. The surface of the film can be smooth (see FIG. 1A), or the film surface can be partially or completely micro-textured. Suitable surface textures include micro-grooves or micro-posts, as shown, for instance, in FIGS. 1B and 1C. See, also, FIGS. 2A, 2B and 2C depicting biodegradable polyester films plated onto both smooth (FIG. 1A) and micro-textured films (mico-grooves shown in FIG. 2B and mico-posts shown in FIG. 3B). The micro-textures can be formed using polyester molding and film forming techniques well know in the art. The film can be cut and shaped to form a suitable shape for implantation.

The film is seeded or plated with retinal progenitor cells. The primary source of the retinal progenitor cells, in one aspect, can be pre-natal retinal tissue. Isolated human retinal progenitor cells can be derived by the dissection of the human neural retina from host tissue, e.g., a living host or a cadaver, prenatal sources, fetal tissue or adult tissue, and can be isolated from the retinal neurosphere. The cells can also be identified by markers, that include, for example, Otx2, Sox2, Pax6—eye field development transcription factors; CyclinD1, Ki67, hTERT—proliferative markers; cMyc, Klf4, Oct4—“sternness” transcription factors; SSEA4—surface antigen, characteristic for undifferentiated cells. Preferably, the retinal progenitor cells express both HIF1 and HIF2.

During dissection, it may necessary to manage the highly tenacious vitreous gel component. This can be accomplished using a variety of techniques, alone or in combination, including vitrectomy, ocular inversion, mechanical resection and absorbent debridement, as well as enzymatic digestion. Suitable enzymes for this purpose include, but are not limited to, hyaluronidases and collagenases. It may also be advantageous to remove non-neural retinal tissue from the specimen used for retinal stem cell isolation. The non-neural tissue includes the optic nerve head and epithelium of the pars plana of the ciliary body, which is typically adherent along the peripheral margin (ora serrata). The tissue is preferably handled using aseptic techniques.

The isolated neuroretinal tissue can be mechanically macerated, and passed through a nylon mesh screen of about 100 micron pore size to dissociate the isolated neuroretinal tissue into cells. The use of a sterile small pore filter screen for the mechanical dissociation of the tissue permits the minimization of the use of enzymes that can degrade cell surface molecules such as growth factor receptors.

An aliquot of cells from the dissected tissue can then be placed in a culture vessel, such as a plastic tissue culture flask, which is preferably coated with a protein layer. Advantageously, the layer may be polyornithine overlaid with laminin or fibronectin.

The aliquot of cells can then be incubated, if preferred, in a first cell culture medium to provide an initial cell concentration for about 24 hours at about 35° C.-39° C., in low oxygen conditions (1% to 6%, preferably 2% to 4%, and most preferably 3% in the culture media). The first cell culture medium can include a physiologically balanced salt solution containing a D-glucose content of from about 0.5-3.0 mg/liter, preferably about 1 mg/liter, N₂ Supplement, as well as 5-15% by volume neural/retinal-conditioned media and an effective amount of at least one antibiotic, such as gentamycin.

After about 24 hours of incubation in the first culture medium, that medium can be removed from the culture vessel. The second culture medium can include a physiologically balanced salt solution containing a glucose content of about 0.5-3.0 mg/liter, preferably 1 mg/liter (e.g., Ultraculture media), at least one growth factor at a concentration of about 30-50 ng/ml per growth factor, an effective amount of L-glutamine (about 0.5-3.0 mM, preferably about 1.0 mM), an effective amount of neural progenitor cell-conditioned medium, and an effective amount of at least one antibiotic, such as penicillin and/or streptomycin, in a low oxygen concentration as described previously. Advantageously, penicillin and/or streptomycin may be added as follows: 10,000 units/ml pen, 10,000 microgram/ml strep, added 1:50-150, preferably 1:100, for a final concentration of 100 units/ml, 100 microgram/ml, respectively, in the culture medium. Those of ordinary skill in the art reading this specification will appreciate that minor modifications can be made to the design of the culture media components and operating conditions. See, also, co-pending U.S. application Ser. No. 13/160,002, filed Jun. 14, 2011, the full disclosure of which is incorporated by reference herein.

The retinal progenitor cells can be plated directly onto the biodegradable polymer film to foam a biocompatible scaffold. Alternatively, the polymer film can be coated with a suitable coating material such as poly-D-lysine, poly-L-lysine, fibronectin, laminin, collagen I, collagen IV, vitronectin and matrigel. The cells can be plated to any desired density, but a single layer of cells (a monolayer) is preferred. The cells can be further propagated after plating, either in vitro where the cells can be harvested, or in vivo following transplantation into the sub-retinal space of a human eye. The retinal progenitor cells of the invention are multipotent and capable of differentiating into specialized retinal cells, particularly photoreceptor cells.

### Therapeutic Use

This invention also provides methods for replacing or repairing photoreceptor cells in a patient in need of this treatment comprising implanting the biocompatible scaffold described above in a sub-retinal space of a diseased or degenerated human retina. In one aspect, the biocompatible scaffold can treat or alleviate the symptoms of retinitis pigmentosa in a patient in need of the treatment. In another aspect, the biocompatible scaffold can treat or alleviate the symptoms of age related macular degeneration in a patient in need of this treatment. For all of these treatments, the retinal progenitor cells can be autologous or allogeneic to the patient. In a further aspect, the cells and scaffolds of the invention can be administered in combination with other treatments.

### Screening Assays

The present invention provides methods for screening various agents that modulate the differentiation of a retinal progenitor cell. It could also be used to discover therapeutic agents that support and/or rescue mature photoreceptors that are generated in culture from retinal progenitor cells grown on the polymer scaffolds. For the purposes of this invention, an “agent” is intended to include, but not be limited to, a biological or chemical compound such as a simple or complex organic or inorganic molecule, a peptide, a protein (e.g. antibody), a polynucleotide (e.g. anti-sense) or a ribozyme. A vast array of compounds can be synthesized, for example polymers, such as polypeptides and polynucleotides, and synthetic organic compounds based on various core structures, and these are also included in the term “agent.” In addition, various natural sources can provide compounds for screening, such as plant or animal extracts, and the like. It should be understood, although not always explicitly stated, that the agent is used alone or in combination with another agent, having the same or different biological activity as the agents identified by the inventive screen.

To practice the screening method in vitro, an isolated population of cells can be obtained as described above. When the agent is a composition other than a DNA or RNA, such as a small molecule as described above, the agent can be directly added to the cells or added to culture medium for addition. As is apparent to those skilled in the art, an “effective” a mount must be added which can be empirically determined. When the agent is a polynucleotide, it can be directly added by use of a gene gun or electroporation. Alternatively, it can be inserted into the cell using a gene delivery vehicle or other method as described above. Positive and negative controls can be assayed to confirm the purported activity of the drug or other agent.

The invention may be further described and illustrated in the following examples which are not in tended to limit the scope of the invention thereby.

### EXAMPLES

### Materials and Methods

### Retina Morphology

The morphology of the neural retina which is the subject of this invention is further described in commonly assigned co-pending U.S. application Ser. No. 13/160,002, filed Jun. 14, 2011, the full disclosure of which is incorporated by reference herein.

### Cell Isolation

hRPCs (human retinal progenitor cells) were isolated from fetal retina as described, with small modifications, in the following references: Klassen, H. J. et al., Multipotent Retinal Progenitors Express Developmental Markers, Differentiate into Retinal Neurons, and Preserve Light-Mediated Behavior, Invest. Opthalmol. Vis. Sci., 2004, 45(11), pages 4167-4173; Klassen, H. et al., Isolation of Retinal Progenitor Cells from Post-Mortem Human Tissue and Comparison with Autologous Brain Progenitors, J. Neuroscience Research, 2004, 77(3), pages 334-343; Klassen, H. et al., Progenitor Cells from the Porcine Neural Retina Express Photoreceptor Markers after Transplantation to the Subretinal Space of Allorecipients; Stem Cells, 2007, 25(5); pages 1222-1230. Briefly, whole neuroretinas from human fetal eyes (14-18 weeks gestational age) were dissected, dissociated in 0.1% collagenase I (Sigma) during 4 cycles (1.5 hour of fermentation in total), and plated in modified Ultraculture media (10 ng/ml rhEGF, 20 ng/ml rhbFGF, Pen/strep, Nystatin and L-glutamine) or frozen. The amount and viability of single cells and clumps were estimated using Trypan blue and a haemocytometer.

### Cell Culture

Cells were plated at a density of approximately 10,000 cells/cm² and cultured under physiologic oxygen (3% oxygen) conditions at 37° C., 100% humidity, 5% CO² in modified Ultracutlure media (10 ng/ml rhEGF, 20 ng/ml rhbFGF, Pen/strep, Nystatin and L-glutamine). Cells were plated on an 8-well slide control, and on polystryrene (nonbiodegradable) and polycaprolactone (biodegradable) thin films (approximately 5 μm in thickness). The slides and polymer films were coated with fibronectin prior to plating the cells. Cells were passaged at 75%-85% confluence (usually each 2-5 days) using Trypsin-EDTA solution. At each passage, the cells were counted and plated at the density mentioned above. Low-oxygen conditions were created in a Thermo 150i incubator, not exceeding the limit of 6% oxygen.

### Cell Proliferation and Growth Curve

Cell proliferation was assessed during routine passaging by cell count via a haemocytometer (at least in two flasks for each passage/source). CyQuant NF assay (Invitrogen) was performed to estimate proliferation speed on each passage: a calibration curve was built by plating 1000, 2000, 4000 and 8000 cells in wells of a 96-well plate (BD Optilux). The amount of cells in experimental wells (4000 cells/well) was assessed by CyQuant staining after 48 and 72 hours (n=4 for calibration curve and n=6 for experimental wells).

### Apoptosis

hRPCs (p1-p9) for TUNEL assay (Roche) were plated in 8-well slides coated with fibronectin, the same way as for maintenance conditions (4,000 of alive cells in each well, hRPC media with supplements); 48 hours after plating cells were fixed, permeabilised (0.01% Triton-X, 0.01% sodium citrate), and stained for double-stained DNA breaks. Slides were mounted, and a cell count was performed in 9 fields of view for each condition. Western blot analysis for pro-survival pathway proteins p44/42 and p38 (Cell Signaling) was performed (protein was collected after 4 days in culture).

### Immunocytochemistry

Cells were assessed via immunocytochemical analysis for sternness and proliferation marker expression: Otx2, Sox2, Pax6—eye field development transcription factors; CyclinD1, Ki67, hTERT—proliferative markers; cMyc, Klf4, Oct4—“sternness” transcription factors; SSEA4—surface antigen, characteristic for undifferentiated cells. For this purpose, 4,000 cells were plated in each well of 16-well fibronectin coated chamber glass slides (Nunc). After 24 hours of incubation under appropriate conditions, cells were washed in PBS, fixed (cold, freshly prepared 4% PFA), permeabilised (0.02% Triton X-100 in 5% BSA), blocked and stained with primary antibodies overnight at 4° C., and secondary antibodies (1:50, Goat Cy3-conjugated anti-rabbit or anti-mouse, Jackson Immunoresearch) for 1 hr at room temperature.

### Western Blot

hRPCs cultured under the conditions described above for 4 days were harvested for protein analysis on passages 1, 3, 5, 7, 10 and 16, lysed in RIPA buffer, and analyzed for protein expression by Western blot. Proteins were separated on 8% SDS-PAGE gel, transferred to a PVDF membrane (Bio-Rad), which was blocked with 5% non-fat milk (Bio-Rad) in TBS-T, and stained with antibodies diluted in 5% BSA in TBS-T (EGFR, HIF1alpha, HIF2alpha, hTERT, Nestin, Sox2, Oct4, Klf4, cMyc, p44/42, and p38). Resulting bands were imaged with ECL Plus (Perkin Elmer) and CL-Xposure film (Thermo Scientific). Anit-bActin HRP-linked antibodies (Abcam) were used as a loading control. Band square was measured using ImageJ.

### Telomerase Activity Assay

Telomerase activity was assessed by the TRAPeze method according to the manufacturer's (Millipore) instructions. Briefly, cells were harvested, lysed in CHAPS buffer for 30 minutes on ice, and the telomers were amplified for 30 minutes at 30° C. The products were amplified using Platinum Taq (Invitrogen), separated by PAGE gel electrophoresis (non-reducing conditions) and stained with SYBR Gold (1:10000, Invitrogen) for 20 minutes at room temperature.

### Differentiation Abilities In Vitro

To assess the ability of hRPCs to differentiate in vitro, hRPCs expanded in 3% oxygen were plated from passages 1, 5, 10 and 16 on fibronectin & laminin-coated 16-well slides. The cells were cultured in differentiating media (DMEM/F12, 1XNEAA, L-glu, 5% HI FBS, Pen/strep and Nystatin) in 3% oxygen. On days 2, 5 and 9, cells were fixed and stained for blue opsin (short-wavelength cones), red/green opsin (long-wavelength cones), rhodopsin (rods), recoverin (photoreceptor precursor), calbindin (horizontal cells), GFAP (Muller & ganglion cells), Glutamine sythetase (ganglion cells), MAP2 and Cyclin D3 (gangion cells) and PKCa (bipolar cells). The same staining was performed for hPRC on the same passages but after 24 hours in maintenance conditions. The ability to differentiate was estimated by comparing the number of cells expressing mature retinal markers in differentiating versus maintenance conditions.

### Results

A biodegradable scaffold was constructed with polycaprolactone (PCL) film coated with fibronectin. For comparison and evaluation, the films had various surface topographies, ranging form smooth to micro-textures in the form of micro-grooves and micro-posts. Human retinal progenitor cells as described herein were plated onto the films. The cells were isolated from a human retina at 14 weeks to 18 weeks gestational age and expanded in vitro in low-tension oxygen (3%). At passage 5, the cells were seeded in an 8-well slide as a control, and on PCL scaffolds having the three different surface characteristics as described. Cells were also plated and grown on a polystyrene film (non-biodegradable) as a control.

After one week following the initial preparation of the cells and scaffolds, real time polymerase chain reaction (PCR) and immunocytochemistry (ICC) assays were performed on the cells cultured on the biodegradable polymer, the polystyrene polymer and the control cells. The differentiation of the retinal progenitor cells into photoreceptor cells was evaluated. Explant experiments and sub-retinal transplantation of pre-differentiated retinal progenitor cells was performed to analyze the cell migration and integration into the host retina of rhodopsin knockout mice.

Summarizing the results obtained, cells grown on polycaprolactone films had enhanced attachment, organization and proliferation as compared to both control cells and cells grown on polystyrene films. Cells grow on micro-textured PCL films were additionally enhanced as compared to cells grown on smooth PCL films. Human retinal progenitor cells adhered to PCL films were observed to differentiate toward mature photoreceptor phenotypes as evidenced by changes in mRNA and protein levels. Using real time quantitative PCR and ICC, a statistically significant upregulation in the expression of rhodopsin, CRX and recoverin, and a statistically significant downregulation of SOX2 (a marker for undifferentiated progenitor cells) and PAX6, also compared to the control cells and cells grown on polystyrene, was observed.

Using flow cytometry and ICC, a significant increase in the ratio of cells expressing photoreceptor markers was observed after 7 days culture in Ultraculture media supplemented with bFGF and EGF (PCL coated with fibronectin vs. control). These markers included Rhodopsin (45% vs. 5%), Opsin Red/Green (20% vs. 3%), Opsin Blue (20% vs. 5%). As a result of the high ratio of differentiated cells, better integration of cells was observed on PCL films with different micro-topography independent of film thickness. Transplanted pre-differentiated human retinal progenitor cells were observed to migrate into the outer nuclear layers of the host retina and exhibit photoreceptor mature marker expression.

### Cell Morphology

The cell morphology was evaluated for cells plated on polycaprolactone films coated with fibronectin. The morphology was observed to be different for smooth and micro-textured surfaces as shown in FIGS. 2A, 2B and 2C. The difference in microtopography of the film surface did not alter the differentiation of the retinal progenitor cells.

### Cell Proliferation and Growth Curve

The rate of hRPC (human retinal progenitor cell) proliferation was measured for polycaprolactone (PCL) films having smooth, micro-grooved and micro-post surface topography. These results are shown in FIG. 3 which is a graph of the cell density against time (days). The highest cell density was observed for cells deposited on polycaprolactone films having micro-grooves.

### Proliferative Markers

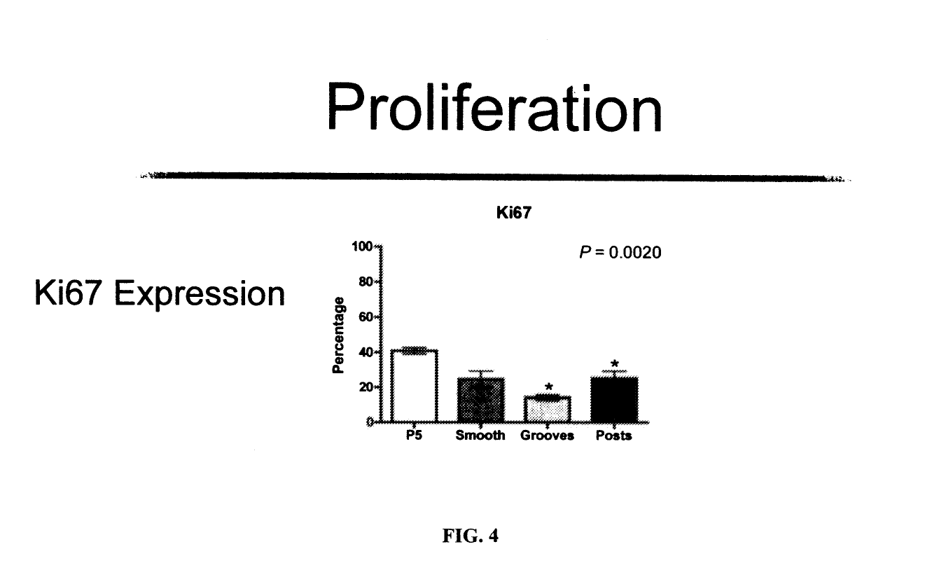

The observed decrease in hRPC proliferation for cells plated on PCL correlates with the decrease in expression of markers Ki67. See FIG. 4 depicting bar graphs for proliferative marker Ki67 for the control (P5) and cells plated on PCL films having varying surface micro-textures as shown.

### Differentiation

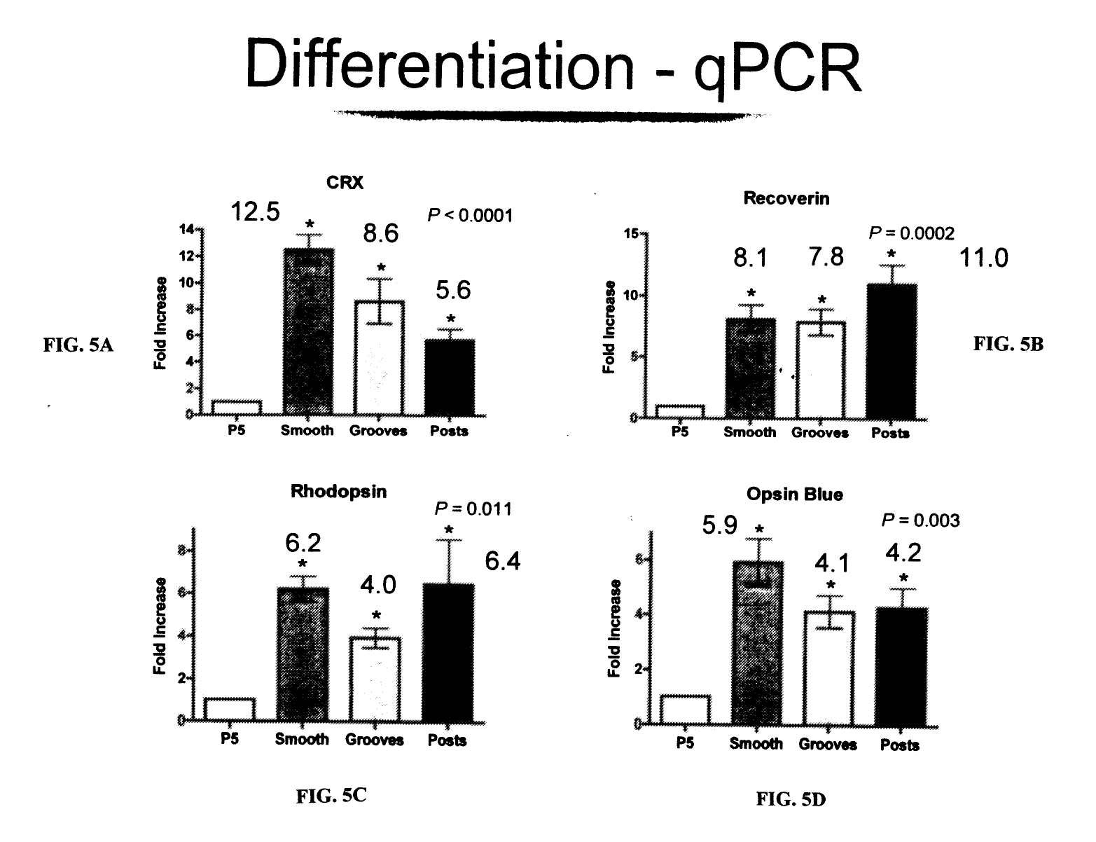

The main characteristics of hRPC cells are functional—the ability to differentiate into specialized retinal cells. Differentiation markers were analyzed using qPCR for cells plated on various PCL substrates and control cells. FIGS. 5A-5D show the results for various differentiation markers: FIG. 5A (CRX); FIG. 5B (Recoverin); FIG. 5C (Rhodopsin); and FIG. 5D (Opsin Blue).

### Sternness Markers

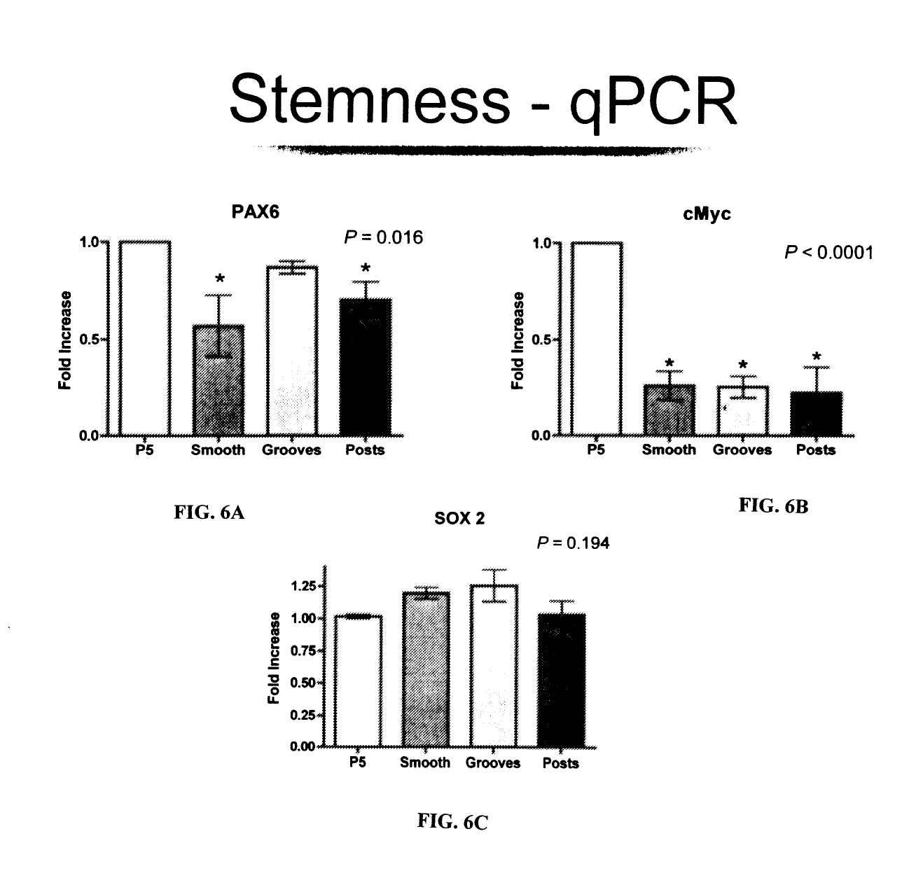

The following sternness markers were evaluated using qPCR for cells plated on various PCL substrates and control cells. FIGS. 6A-6C show the results for the sternness markers: FIG. 6A (Pax6); FIG. 6B (cMyc); FIG. 6C (Sox2).

The use of other polymers as scaffolds for retinal progenitor cells has not proven effective. Such polymers include polystyrene, polymethyl methacrylate (PMMA), and polyglycerol sebacate (PGS). While some of these polymers are biodegradable (PGS), and other can include topographical features (PMMA), none of these polymers were found to be capable of providing a satisfactory platform for the differentiation of retinal progenitor cells into viable photoreceptors.

The human retinal progenitor cells and biodegradable scaffolds of this invention may be used for studying the development of the retina and eye, as well as factors affecting such development, whether beneficially or adversely. These hRPCs can also be used for clinical trials by transplantation into a suffering retina from dysfunctions of the eye. They may be used advantageously to repopulate or to rescue a dystrophic and degenerated ocular tissue, particularly a dysfunctional retina. Retinal dysfunction encompasses any lack or loss of normal retinal function, whether due to disease, mechanical or chemical injury, or a degenerative or pathological process involving the recipient's retina. The hRPCs may be injected or otherwise placed in a retinal site, the subretinal space, vitreal cavity, or the optic nerve, according to techniques known in the art.

Advantageously, the hRPCs of the invention may be used to compensate for a lack or diminution of photoreceptor cell function. Examples of retinal dysfunction that can be treated by the retinal stem cell populations and methods of the invention include but are not limited to: photoreceptor degeneration (as occurs in, e.g., retinitis pigmentosa, cone dystrophies, cone-rod and/or rod-cone dystrophies, and macular degeneration); retina detachment and retinal trauma; photic lesions caused by laser or sunlight; a macular hole; a macular edema; night blindness and color blindness; ischemic retinopathy as caused by diabetes or vascular occlusion; retinopathy due to prematurity/premature birth; infectious conditions, such as, e.g., CMV retinitis and toxoplasmosis; inflammatory conditions, such as the uveitis; tumors, such as retinoblastoma and ocular melanoma; and for the replacement of inner retinal neurons, which are affected in ocular neuropathies including glaucoma, traumatic optic neuropathy, detachment, and radiation optic neuropathy and retinopathy.

The treatments described herein can be used as stand alone therapies, or in conjunction with other therapeutic treatments. Such treatments can include the administration of a substance that stimulates differentiation of the neuroretina-derived stem cells into photoreceptors cells or other retinal cell types (e.g., bipolar cells, ganglion cells, horizontal cells, amacrine cells, Mueller cells).

From the foregoing, it will be appreciated that, although specific embodiments of the invention have been described herein for purposes of illustration, various modifications may be made without deviating from the spirit and scope of the invention as set forth in the appended claims. All publications, patents, and patent applications referenced herein are incorporated by reference in their entirety.

1. A biocompatible composition comprising:
a biodegradable polyester carrier film, the surface of said film being micro-textured, and
a layer of isolated and substantially homogenous undifferentiated human retinal progenitor cells adhered to at least a portion of the surface of said polyester film,
wherein said undifferentiated cells are capable of differentiating into photoreceptor cells.

2. The composition of claim 1 wherein the polyester is selected from the group consisting of polylactic acid (PLA), polycaprolactone (PCL), polyesteramide (PEA), polyhydroxybutyrate (PHB), and derivatives and mixtures thereof.

3. The composition of claim 1 wherein the polyester is polycaprolactone (PCL).

4. (canceled)

5. (canceled)

6. The composition of claim 1 wherein the differentiated photoreceptor cells are transplanted into a host.

7. The composition of claim 1 wherein the differentiated photoreceptor cells are used for drug discovery and testing.

8. The composition of claim 1 wherein the differentiated photoreceptor cells are cultured in the presence of at least one exogenous growth factor.

9. The composition of claim 3 which is a biocompatible scaffold for human retinal transplantation into a human eye.

10. The composition of claim 1 wherein a coating of is applied to the polyester film surface prior to depositing human retinal progenitor cells onto the film.

11. The composition of claim 10 wherein the coating is a material selected from the group consisting of poly-D-lysine, poly-L-lysine, fibronectin, laminin, collagen I, collagen IV, vitronectin, matrigel, and mixtures thereof.

12. The composition of claim 1 wherein the retinal progenitor cells are obtained from post-natal retinal tissue.

13. The composition of claim 1 wherein the retinal progenitor cells are obtained from the fetal neural retina.

14. The composition of composition 1 wherein the polyester film has a thickness in the range of from about 1.0 μm to about 10 μm, and preferably about 5 μm.

15. The composition of claim 1 wherein the layer of cells is a monolayer.

16. (canceled)

17. The composition of claim 1 wherein the texture is in the form of a series of micro-grooves.

18. The composition of claim 1 wherein the texture is in the form of a series of micro-posts.

19. A method for drug discovery comprising
contacting a candidate drug target with human retinal progenitor cells obtained in claim 4,
evaluating the interaction of the drug target with said cells, and
selecting a viable drug candidate based on said interaction.

20. The method of claim 19 wherein said interaction involves enhanced proliferation and/or differentiation of said retinal progenitor cells.

21. A method for treatment of a diseased or degenerated human retina in a patient comprising transplanting the composition of claim 3 into a sub retinal space of a human eye to thereby replace or repair photoreceptor cells in said patient.

22. The method of claim 21 wherein the diseased or degenerative condition is selected from the group consisting of retinis pigmentosa, age related macular degeneration, traumatic optic neuropathy and retina detachment.

23. The method of claim 21 wherein the diseased or degenerative condition is age related macular degeneration.
